(19) United States
(12) Patent Application Publication
(10) Pub. No.: US 2019/0167722 A1
(43) Pub. Date: Jun. 6, 2019

(54) TRANSFECTION OF DENDRITIC CELLS AND METHODS THEREFOR

(71) Applicants: Nant Holdings IP, LLC, Culver City, CA (US); Nantcell, Inc., Culver City, CA (US)

(72) Inventors: Patrick Soon-Shiong, Culver City, CA (US); Kayvan Niazi, Culver City, CA (US)

(21) Appl. No.: 16/322,310

(22) PCT Filed: Aug. 2, 2017
(86) PCT No.: PCT/US2017/045093

§ 371 Date: Jan. 31, 2019

Related U.S. Application Data
(60) Provisional application No. 62/370,208, filed on Aug. 2, 2016.

(51) Int. Cl. A61K 35/17; C12N 5/0783; A61K 35/15; A61K 38/20; A61K 39/00; C12N 5/0781; C12N 5/0784

(52) U.S. Cl. CPC A61K 35/17; A61K 2039/5158; A61K 35/15; A61K 38/2013; A61K 39/0011; C12N 5/0635; C12N 5/0639; A61K 38/2046; A61K 38/208; A61K 38/2086; C12N 2502/1107; C12N 2502/1114; C12N 2502/1121; C12N 2502/1157; C12N 2502/1164; C12N 5/0636

(57) ABSTRACT

Immunotherapeutic methods and compositions are contemplated where one or more neoepitopes and/or tumor associated antigens are produced in, or delivered to dendritic cells, and in which so modified dendritic cells are co-cultured with immune competent cells of a patient, preferably in the presence of stimulatory signals. Cells are then transfused to the patient that has preferably undergone immune checkpoint inhibition treatment.

This application claims priority to U.S. provisional application with the Ser. No. 62/370208, filed 2 Aug. 2016.

### FIELD OF THE INVENTION

The field of the invention is immunotherapeutic compositions and methods, especially as it relates to cancer vaccine preparations and methods having an ex vivo component.

### BACKGROUND

The following description includes information that may be useful in understanding the present invention. It is not an admission that any of the information provided herein is prior art or relevant to the presently claimed invention, or that any publication specifically or implicitly referenced is prior art.

All patent applications and publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer vaccines have shown much promise in recent years, but are often limited due to various factors, including immunogenicity of a viral vehicle and poor presentation of the recombinant antigen. Moreover, due to often systemic delivery of viral vehicles, pervasive training of the various components in the immune system (e.g., dendritic cells, CD8+ T-cells, CD4+ helper T-cells, B-cells) is often not or only poorly achieved. Furthermore, even where antigen presentation is achieved to at least some degree, immune checkpoint inhibition will often present an additional hurdle to effective treatment.

To overcome some of the difficulties that are associated with the systemic delivery of tumor antigens, various efforts have been undertaken to trigger in vitro stimulation of certain antigen presenting cells using cancer specific antigens. The so pulsed antigen presenting cells are then transfused to a patient as a therapeutic composition (see e.g., Nature Reviews|Cancer 2012 Vol. 12 p. 265-277). In another approach, dendritic cells were incubated with NK cells to generate mature dendritic cells in the presence of TLR agonists (see e.g., Experimental & Molecular Medicine (2010), 42(6), p 407-419). While at least conceptually attractive, various drawbacks nevertheless remain. Among other things, pulsed dendritic cells are often exposed to tumor cells that carry an inherent risk of generating autoimmunity, or combined with NK cells that frequently lack the potential to elicit a durable immunity.

Thus, even though numerous methods and compositions to generate an immune response are known in the art, all or almost all of them suffer from various disadvantages. Consequently, there remains a need for improved compositions and methods for immunotherapy.

### SUMMARY OF THE INVENTION

The inventive subject matter is directed to compositions and methods of immunologic tumor treatment of a patient in which immune competent cells of a patient (e.g., NK cells, CD4+ T-cells, etc.) are co-cultured with various antigen-presenting cells (e.g., dendritic cells) that were previously transfected with one or more tumor-related epitopes of a tumor of the patient, or that were previously transfected with an expression vector that includes a nucleic acid encoding one or more tumor-related epitopes of the tumor of the patient. The so generated cell population has specific immune reactivity against the tumor-related epitopes of the tumor of the patient and is suitable as a therapeutic modality.

In one aspect of the inventive subject matter, the inventors contemplate a method of treating a patient having a tumor that includes a step of administering to the patient a plurality of immune competent cells that were previously exposed to transfected antigen-presenting cells.

Most preferably, the antigen-presenting cells were transfected with at least one tumor-related epitope of the tumor of the patient or with an expression vector comprising a nucleic acid that encodes the at least one tumor-related epitope of the tumor of the patient, and the immune competent cells were obtained from the patient having the tumor.

Therefore, the inventors also contemplate a method of ex vivo activating immune competent cells from a patient having a tumor. Such methods will typically include a step of obtaining from the patient a plurality of immune competent cells, and a further step of ex vivo transfecting a plurality of antigen-presenting cells with at least one tumor-related epitope of the tumor of the patient or with an expression vector comprising a nucleic acid that encodes the at least one tumor-related epitope of the tumor of the patient. In yet another step, the plurality of immune competent cells are co-cultured with the plurality of transfected antigen-presenting cells for a time sufficient to activate the immune competent cells.

Consequently, and in yet another aspect of the inventive subject matter, the inventors also contemplate a pharmaceutical composition that includes a pharmaceutically acceptable carrier for transfusion in combination with a plurality of immune competent cells and a plurality of transfected antigen-presenting cells. Viewed from a different perspective, the inventors therefore also contemplate the use of a plurality of immune competent cells and transfected antigen-presenting cells to formulate a pharmaceutical composition for the treatment of a tumor of a patient. The antigen-presenting cells are typically cells that were previously transfected with at least one tumor-related epitope of a tumor of a patient or a viral vector comprising a nucleic acid that encodes the at least one tumor-related epitope of the tumor of the patient, wherein the immune competent cells were previously obtained from the patient having the tumor.

While not limiting to the inventive subject matter, it is generally preferred that the immune competent cells are or comprise a white blood cell fraction of the patient's whole blood. For example, suitable immune competent cells may be a collection of white blood cells that are enriched in at least one of a CD4+ T-cell, a CD8+ T-cell, an NK cell, a macrophage, a monocyte, and a B-cell. Similarly it is contemplated that the antigen-presenting cells are from the patient, and most preferably dendritic cells.

In some embodiments, the antigen-presenting cells are transfected with at least one tumor-related epitope of the tumor of the patient, while in other embodiments, the antigen-presenting cells are transfected with an expression vector comprising a nucleic acid that encodes the at least one tumor-related epitope. Preferably, the tumor-related epitope will comprises a tumor neoepitope, a tumor-specific antigen, and/or a tumor associated antigen, and is an HLA-matched tumor-related epitope where desired. Moreover, the tumor-related epitope may further comprise a targeting sequence that targets the tumor-related epitope for MHC-I and/or MHC-II presentation. Of course, it should be appreciated that the antigen-presenting cells may be transfected with at least two distinct tumor-related epitopes of the tumor of the patient or that the nucleic acid encodes at least two distinct tumor-related epitopes of the tumor of the patient.

To enhance immune response, the antigen-presenting cells may be further transfected with or exposed to one or more immune stimulating molecules, or the nucleic acid may further encode at least one immune stimulating molecule, and especially a co-stimulatory molecule (e.g., B7.1 (CD80), B7.2 (CD86), ICAM-1 (CD54), ICOS-L, LFA-3 (CD58), 4-1BBL, CD30L, CD40, CD40L, CD48, CD70, CD112, CD155, GITRL, OX40L, or TL1A). Likewise, the antigen-presenting cells may also be transfected with or exposed to one or more checkpoint inhibitors, or the nucleic acid may further encode one or more checkpoint inhibitors (e.g., polypeptide that binds to CTLA-4 (CD152) or PD-1 (CD 279)).

In further contemplated aspects, the expression vector may be a viral vector, and preferably an adenoviral vector, optionally having a deleted or non-functional E2b gene. Viewed from another perspective, the viral vector may have reduced immunogenicity relative to a corresponding wild-type viral vector.

Where desired, the immune competent cells may also be exposed to the transfected antigen-presenting cells in the presence of a cytokine, for example, IL-2, IL-7, IL-12, IL-15, or a IL-15 superagonist. In addition, it is contemplated that an immune checkpoint inhibitor may be administered to the patient before the step of administering the plurality of immune competent cells, and/or that the immune competent cells are administered together with the transfected antigen-presenting cells. If desired, a viral vector may be administered to the patient that comprises the nucleic acid that encodes one or more tumor-related epitope of the tumor of the patient.

Various objects, features, aspects and advantages of the inventive subject matter will become more apparent from the following detailed description of preferred embodiments.

### DETAILED DESCRIPTION

The inventive subject matter is drawn to various compositions, methods, and uses for immunotherapy, and particularly cell-based compositions, methods, and uses in which one or more types of immune competent cells of a patient having a tumor are exposed ex vivo to dendritic cells that were previously transfected with or exposed to one or more tumor-related epitopes of the tumor of the patient, or that were previously transfected with an expression vector that includes a nucleic acid that encodes one or more tumor related or tumor specific epitopes of the tumor of the patient. In such manner, the immune response can be specifically directed to a particular tumor (and even tumor sub-population), and the immune competent cells of the patient will not be subject to rejection. Moreover, activation of the dendritic cells and instruction of immune competent cells by the dendritic cells can be further enhanced by exposure of the cell mixture to immune stimulating compositions, and particularly to IL-15 (or an IL-15 superagonist).

For example, in one contemplated aspect of the inventive subject matter, immune competent cells of a patient diagnosed with a colon cancer are isolated, typically in form of a white cell fraction of whole blood (e.g., isolated as buffy coat). From this fraction, or another sample of the patient (e.g., from skin or spleen), dendritic cells are isolated. Alternatively, dendritic cells may also be derived from progenitor cells in response to specific growth factors (e.g., GM-CSF). Regardless of the type of isolation, it is then contemplated that the dendritic cells are transfected with one or more tumor-related epitopes of the tumor of the patient or with an expression vector (preferably viral vector) that includes a nucleic acid that encodes the one or more tumor-related epitopes of the tumor of the patient. Transfection is preferably performed using known transfection agents, or mechanically induced transfection. Most preferably, and as is further discussed in more detail below, the tumor-related epitopes include or are neoepitopes specific to the patient's tumor. As a result, the so transfected dendritic cells will present the tumor epitopes via the MHC-I/MHC-II system.

Advantageously, the dendritic cells that present the tumor epitopes are contacted ex vivo with the previously isolated immune competent cells (or white blood cell fraction), preferably in the presence of an immune stimulatory cytokine (e.g., IL-2, IL-7, IL-15 or IL-15 superagonist). As a result of the presentation of the tumor-related epitopes by the dendritic cells, the immune competent cells will be activated and the so activated immune competent cells can then be transfused to the patient, typically with the dendritic cells. However, it should be noted that it is not deemed necessary to remove other components from the activated immune competent cells, and that the co-cultured cells can be administered directly to the patient. Still further, it should be appreciated that as the so obtained immune therapeutic composition is derived from the patient, no rejection reaction should be observed. In addition, stimulation with immune stimulatory cytokines is limited to the ex vivo step and as such should not lead to the otherwise undesirable side effects of systemic administration. To further augment the immune response in the patient, it is contemplated that the patient may undergo treatment with one or more immune checkpoint inhibitors (e.g., ipilimumab, pembrolizumab, nivolumab) before and/or during administration of the activated immune competent cells.

Of course, it should be appreciated that the compositions and methods presented herein are not limited to patients diagnosed with colon cancer, and that indeed all conditions are contemplated that associated with an incomplete, suppressed, or lacking immune response against an otherwise proper antigen. Therefore, contemplated alternative diseases include various other solid and blood borne cancers, including breast cancer, pancreatic cancer, liver cancer, gastric cancer, lung cancer glioblastoma, melanoma, lymphoma, etc.

Likewise, contemplated immune competent cells of a patient diagnosed with cancer need not be limited to the white blood cell fraction/buffy coat of the patient's whole blood, but may include fractions enriched in one or more of CD4+ T-cells, CD8+ T-cells, NK cells, monocytes, macrophages, and B-cells. Of course, it should be noted that such cells may be isolated to relatively high purity (e.g., at least 80%, more typically at least 90%, most typically at least 95%). However, in less preferred aspects, the immune competent cells may also be provided as a whole blood sample. Moreover, it should be appreciated that the immune competent cells may also be provided from an HLA-matched non-patient donor, where the HLA match is an at least 4 digit match for one or more (and typically all) of HLA-A, B, C, DRB1/B3/B4, and DQB1 loci by standard methods such as PCR-SSO assay on microbeads arrays. Likewise, immune competent cells may also be allogenic and genetically modified (e.g., expressing patient-specific HLA) to have reduced antigenicity.

Most typically, the number of immune competent cells will be in the range of between about 10⁶ to 10¹⁰ cells, or in the range of 10⁶ to 10⁸ cells, or in the range of 10⁷ to 10⁹ cells, or in the range of 10⁸ to 10¹⁰ cells, or even higher. Depending on the type of immune competent cells, it should be appreciated that the immune competent cells may be cultured to expand in number, combined to achieve a specific ratio (e.g., CD4+ T-cells and CD8+ T-cells at an about 10:1 ratio to NK cells), or that particular types of immune competent cells may be enriched to accommodate a particular manner of antigen presentation (e.g., CD8+ enriched where antigen presentation is towards MHC-I, or CD4+ enriched where antigen presentation is towards MHC-II). Thus, it should be appreciated that the immune competent cells of the patient may include one, or two, or three, or four, of five, or all of CD4+ T-cells, CD8+ T-cells, NK cells, monocytes, macrophages, and B-cells.

It is still further contemplated that selected immune competent cells, and particularly exhausted T-cells may be removed from the immune competent cells, most typically using magnetic bead separation or FACS separation based on surface markers of T-cell exhaustion. For example, suitable exhaustion markers include CD160, 2B4, LAG3, PD1, TIM3, etc. Such depletion may advantageously increase the overall population of activated T-cells with respect to the antigens presented by the dendritic cells. On the other hand, exhausted T-cells may also be reactivated before contact with the dendritic or other antigen presenting cells, for example, using various compounds such as IL21, or antibodies against PD-L1, TIM3, LAG3, or CTLA4.

Likewise, it should be appreciated that the immune competent cells may be exposed to one or more immune stimulatory compounds or compositions before contacting the dendritic or other antigen presenting cells, and suitable immune stimulatory compounds or compositions include various cytokines and chemokines, especially including IL-1, IL-2, IL-15, and IL-21. For example, the immune competent cells may be exposed to IL-2 or IL-15 (e.g., where T-cells are part of the immune competent cells), or TNF-alpha (e.g., where macrophages are part of the immune competent cells) or Interferon-gamma (e.g., where NK cells are part of the immune competent cells) to further stimulate activity of the immune competent cells. Notably, such in vitro immune stimulation can be performed at conditions that would be at least problematic in vivo (e.g., due to vascular leak syndrome where IL-2 is employed). Where desired, the immune stimulatory compounds or compositions may be removed before contacting the immune competent cells with the dendritic or other antigen presenting cells.

Similarly, it should be appreciated that suitable antigen-presenting cells need not be limited to dendritic cells, but that numerous alternative professional and non-professional antigen-presenting cells (and all reasonable mixtures thereof) are also deemed appropriate. Therefore, suitable antigen-presenting cells include dendritic cells, macrophages, B-cells, etc. However, it is noted that dendritic cells are generally preferred. Most typically, the dendritic cells will be isolated from the same patient, for example, from blood, spleen, or skin (see e.g., Curr Protoc Immunol. 2001 May; Chapter 7: Unit 7.32, or J Immunol Methods. 2001 June 1;252(1-2):93-104). However, in alternative aspects, dendritic cells may also be derived from the patient's progenitor cells using suitable factors (e.g., GM-CSF, alpha TNF, or various other cytokines) as is well known in the art (see e.g., Front Microbiol. 2013; 4: 292).

Regardless of the manner of obtaining antigen-presenting cells, it is contemplated that such antigen-presenting cells can be activated or otherwise stimulated before contacting the antigen-presenting cells with the immune competent cells (which may or may not have been exposed to immune stimulatory compounds or compositions). Such stimulation or activation is particularly advantageous where it is desired that the antigen presenting cells have an increased expression (relative to unstimulated or non-activated cells) of one or more co-stimulatory molecules. For example, it is contemplated that the antigen-presenting cells may be exposed to one or more ligands of pattern recognition receptors such as TLR ligands (e.g., TLR2, TLR3, TLR4, TLR5, TLR7/8, TLR9, TLR13, etc.), NLR ligands (e.g., NOD1, NOD2, etc.), RLR ligands (e.g., 5′ppp-dsRNA, Poly(dA:dT, etc.), CLR ligands (e.g., HKCA, lichenan, beta glucan peptide, etc.), and/or STING ligands (e.g., cyclic dinucleotides such as 2′2′-cGAMP, 2′3′-cGAMP, c-di-AMP, etc.). It should be especially appreciated that such in vitro stimulation of the antigen-presenting cells is particularly beneficial as stimulation can be performed under conditions that would otherwise trigger adverse or autoimmune reactions, or be toxic to a patient.

Most typically, the number of antigen presenting cells will be in the range of between about 10⁶ to 10¹⁰ cells, or in the range of 10⁶ to 10⁸ cells, or in the range of 10⁷ to 10⁹ cells, or in the range of 10⁸ to 10¹⁰ cells, or even higher. Moreover, depending on the type of antigen presenting cells, it should be appreciated that the antigen presenting cells may be cultured to expand in number, combined to achieve a specific ratio (e.g., dendritic cells at an about 10:1 ratio to macrophages), or that particular types of antigen presenting cells may be enriched to accommodate a particular manner of transfection or exposure to the tumor-related epitope (e.g., patient and tumor specific neoepitope, cancer associated antigen, or cancer specific antigen). Thus, it should be appreciated that the antigen presenting cells of the patient may include one, or two, all of dendritic cells, macrophages, and B-cells. Of course, it should also be appreciated that the dendritic cells may be of specific origin (e.g., skin, peripheral blood, spleen, etc.).

With respect to suitable ratios of antigen presenting cells (previously transfected with nucleic acid encoding a tumor related antigen or exposed to tumor related antigen) to immune competent cells it is contemplated that the suitable ratios are typically between 10⁴:1 (antigen presenting cells to immune competent cells) and 1:10⁴ (antigen presenting cells to immune competent cells), or between 10³:1 and 1:10³, or between 10²:1 and 1:10², or 10:1 and 1:10. Once combined, it should be recognized that the cells may be further exposed to immune stimulating compounds and compositions as is further discussed in more detail below.

With respect to contemplated tumor related epitopes, it should be noted that the tumor related epitopes may be tumor and patient specific neoepitopes as is further discussed in more detail below, cancer associated antigens (e.g., CEA, MUC1, etc.), and/or cancer specific antigens (e.g., HER2, PSMA, etc.). Thus, it should be noted that the specificity of the immune competent cells may be fine-tuned towards a specific tumor or even sub-clonal population of a tumor using neoepitopes, or that the immune competent cells may be trained towards a broader population of cells of a tumor. Tumor related epitopes are typically part of a larger polypeptide or may be epitopes having a length of between 7-50 amino acids, possibly concatenated with suitable non-immunogenic interspersed spacers. For example, where the epitope is intended for presentation via MHC-I, a typical length of an epitope may be between 7-15 amino acids. On the other hand, where the epitope is intended for presentation via MHC-II, a typical length of an epitope may be between 15-50 amino acids. Between.

[symbol]

Most preferably, the tumor related epitopes will be encoded on an expression vector or RNA that is transfected into the antigen presenting cell. In particularly preferred aspects, the expression vector is a viral vector, and most preferably an adenoviral vector. Where RNA is used to transfect the cells, the RNA may be mono-cistronic, bi-cistronic, or poly-cistronic. In such case, the expression vector or RNA may be delivered to the bacteria or yeast using known transfection methods. However, it should be appreciated that suitable tumor related epitopes may also be added to the antigen presenting cell as recombinant proteins, or as bacterial vaccine or yeast vaccine preparation. Thus, tumor related epitopes may contact the antigen presenting cells directly via contact with the cell surface or via transfection (e.g., via sonoporation, lipofection, ballistic transfer, etc.) that forces the tumor related epitopes into the cytoplasm. Therefore, the term “transfected” as used in conjunction with the antigen-presenting cells and tumor-related epitopes is meant to include exposure of the antigen-presenting cells to the tumor-related epitopes under conditions that allow the tumor-related epitopes to be taken up into the antigen-presenting cells and manipulation of the antigen-presenting cells (e.g., sonoporation, pressure mediated transfection, chemical transfection, etc.) to force or allow passage of the tumor-related epitopes into the antigen-presenting cells.

With respect to specific sequences of tumor related epitopes it should be appreciated that any epitope that is cancer associated (e.g., CEA, MUC-1, etc.), specific to a type of cancer (e.g., PSA, HER2, etc.), and/or patient- and tumor-specific is suitable for use herein, and especially preferred sequences comprise patient- and tumor-specific neoepitopes. It is still further preferred that the epitope is expressed above healthy control (e.g., from non-diseased tissue of the same patient), and that the epitopes include those predicted of binding to the respective binding motifs of the MHC-I and/or MHC-II complex of the patient.

For example, neoepitopes may be identified from a patient tumor in a first step by whole genome analysis of a tumor biopsy (or lymph biopsy or biopsy of a metastatic site) and matched normal tissue (i.e., non-diseased tissue from the same patient), preferably via location guided synchronous alignment of omics information from the tumor and matched normal tissue of the same patient. So identified neoepitopes can then be further filtered for a match to the patient's HLA type to increase likelihood of antigen presentation of the neoepitope. Most preferably, and as further discussed below, such matching can be done in silico. Most typically, the patient-specific epitopes are unique to the patient, but may also in at least some cases include tumor type-specific neoepitopes (e.g., Her-2, PSA, brachyury) or cancer-associated neoepitopes (e.g., CEA, MUC-1, CYPB1). Thus, it should be appreciated that the adenoviral nucleic acid construct (or nucleic acid construct for other delivery) will include a recombinant segment that encodes at least one patient-specific neoepitope, and more typically encode at least two or three more neoepitopes and/or tumor type-specific neoepitopes and/or cancer-associated neoepitopes. Where the number of selected neoepitopes is larger than the viral capacity for recombinant nucleic acids or exceeds practical limits for RNA, multiple and distinct neoepitopes may be delivered via multiple and distinct RNA or recombinant viruses.

With respect to the step of obtaining omics information from the patient to identify one or more neoepitopes it is contemplated that the omics data are obtained from patient biopsy samples following standard tissue processing protocol and sequencing protocols. While not limiting to the inventive subject matter, it is typically preferred that the data are patient matched tumor data (e.g., tumor versus same patient normal), and that the data format is in SAM, BAM, GAR, or VCF format. However, non-matched or matched versus other reference (e.g., prior same patient normal or prior same patient tumor, or homo statisticus) are also deemed suitable for use herein. Therefore, the omics data may be ‘fresh’ omics data or omics data that were obtained from a prior procedure (or even from a different patient).

Regardless of the nature of the reference sequence (e.g., matched normal), it is generally preferred that the reference sequence is used to calculate a plurality of epitopes. Most typically, the epitopes will be calculated to have a length of between 2-50 amino acids, more typically between 5-30 amino acids, and most typically between 9-15 amino acids, with a changed amino acid preferably centrally located or otherwise situated in a manner that improves its binding to MHC. For example, where the epitope is to be presented by the MHC-I complex, a typical epitope length will be about 8-11 amino acids, while the typical epitope length for presentation via MHC-II complex will have a length of about 13-17 amino acids. It is still further preferred that the so calculated epitopes and neoepitopes are then analyzed in silico for their affinity to the patient-specific HLA-type (MHC-I and MHC-II) as further described below in more detail. It should be appreciated that knowledge of HLA affinity for such neoepitopes provides at least two items of valuable information: (a) deletion of an epitope otherwise suitable for immunotherapy can be recognized and immunotherapy be adjusted accordingly so as to not target the deleted epitope, and (b) generation of a neoepitope suitable for immunotherapy can be recognized and immunotherapy be adjusted accordingly so as to target the neoepitope.

With respect to neoepitope in general, it should be appreciated that neoepitopes can be characterized as random mutations in tumor cells that create unique and tumor specific antigens. Therefore, high-throughput genome sequencing should allow for rapid and specific identification of patient specific neoepitopes where the analysis also considers matched normal tissue of the same patient. Notably, as also disclosed in our copending International application WO 2016/164833, very few neoepitopes appear to be required to illicit an immune response and consequently present a unique opportunity for the manufacture of cancer immunotherapies. Moreover, and as further described below, it should be appreciated that the choice of neoepitope is also further guided by investigation of expression levels and sub-cellular location of the neoepitope. For example, where the neoepitope is not or only weakly expressed relative to matched normal (e.g., equal or less than 20% of matched normal expression), the neoepitope may be eliminated from the choice of suitable neoepitopes. Likewise, where the neoepitope is identified as a nuclear protein, the neoepitope may be eliminated from the choice of suitable neoepitopes. On the other hand, positive selection for neoepitopes may require partially extracellular or transmembrane presence of the neoepitope and/or an expression level of at least 50% as compared to matched normal. Expression levels can be measured in numerous manners known in the art, and suitable manners include qPCR, qLCR, and other quantitative hybridization techniques.

It is generally contemplated that genomic analysis can be performed by any number of analytic methods, however, especially preferred analytic methods include WGS (whole genome sequencing) and exome sequencing of both tumor and matched normal sample. Likewise, the computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Identification of expression level can be performed in all manners known in the art and preferred methods include quantitative RNA (hnRNA or mRNA) analysis and/or quantitative proteomics analysis. Most typically, the threshold level for inclusion of epitopes and neoepitopes will be an expression level of at least 20%, and more typically at least 50% as compared to matched normal, thus ensuring that the (neo)epitope is at least potentially ‘visible’ to the immune system. Thus, it is generally preferred that the omics analysis also includes an analysis of gene expression (transcriptomic analysis) to so help identify the level of expression for the gene with a mutation. Viewed from another perspective, transcriptomic analysis may be suitable (alone or in combination with genomic analysis) to identify and quantify genes having a cancer and patient specific mutation. There are numerous methods of transcriptomic analysis know in the art, and all of the known methods are deemed suitable for use herein. Taken the above into consideration, it should therefore be appreciated that a patient sample comprising DNA and RNA from tumor and matched normal tissue can be used to identify specific mutations and to quantify such mutations. Further epitopes, neoepitopes, methods, and systems suitable for use in conjunction with the teachings presented herein are disclosed in International application WO 2016/172722.

Consequently, it should be recognized that patient and cancer specific neoepitopes can be identified in an exclusively in silico environment that ultimately predicts potential epitopes that are unique to the patient and tumor type. So identified and selected neoepitopes can then be further filtered in silico against an identified patient HLA-type. Such HLA-matching is thought to ensure strong binding of the neoepitopes to the MHC-I complex of nucleated cells and the MHC-II complex of specific antigen presenting cells. Targeting both antigen presentation systems is particularly thought to produce a therapeutically effective and durable immune response involving both, the cellular and the humoral branch of the immune system.

HLA determination for both MHC-I and MHC-II can be done using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types as is shown in more detail below. In short, a patient's HLA-type is ascertained (using wet chemistry or in silico determination), and a structural solution for the HLA-type is calculated or obtained from a database, which is then used as a docking model in silico to determine binding affinity of the neoepitope to the HLA structural solution. Suitable systems for determination of binding affinities include the NetMHC platform (see e.g., Nucleic Acids Res. 2008 Jul. 1; 36(Web Server issue): W509-W512.), HLAMatchmaker (http://www.epitopes.net/downloads.html), and IEDB Analysis Resource (http://tools.immuneepitope.org/mhcii/). Neoepitopes with high affinity (e.g., less than 100 nM, or less than 75 nM, or less than 50 nM for MHC-I; less than 500 nM, or less than 300 nM, or less than 100 nM for MHC-I) against the previously determined HLA-type are then selected. In calculating the highest affinity, modifications to the neoepitopes may be implemented by adding N- and/or C-terminal modifications to the epitope to further increase binding of the virally expressed neoepitope to the HLA-type. Thus, neoepitopes may be native as identified or further modified to better match a particular HLA-type.

For in silico prediction of the HLA-type of a patient, the omics data may be analyzed using a colored De Bruijn graph where the edges are k-mers (k=15) having “colors” that identify which input source the k-mer is found in (e.g., reference, normal sample, and/or tumor sample, samples taken at different times or ages, samples from different patient or subject groups, etc.), and where each edge is connected to adjacent edges. Exemplary systems and methods are described in International application WO 2017/035392.

Thus, it should be appreciated that computational analysis can be performed by docking neoepitopes to the HLA and determining best binders (e.g., lowest KD, for example, less than 50 nM). It should be recognized that such approach will not only identify specific neoepitopes that are genuine to the patient and tumor, but also those neoepitopes that are most likely to be presented on a cell and as such most likely to elicit an immune response with therapeutic effect. Of course, it should also be appreciated that thusly identified HLA-matched neoepitopes can be biochemically validated in vitro prior to inclusion of the nucleic acid encoding the epitope as payload into the virus or generation of an RNA encoding the neoepitope(s).

Most preferably, the recombinant nucleic acid(s) encode cancer associated or cancer-specific epitopes, or patient-specific neoepitopes in an arrangement such that the epitopes are directed to MHC-I and/or MHC-II presentation pathways. With respect to routing the so identified and expressed neoepitopes to the desired MHC-system, it should be appreciated that the MHC-I presented peptides will typically arise from the cytoplasm via proteasome processing and delivery through the endoplasmatic reticulum. Thus, expression of the epitopes intended for MHC-I presentation will generally be directed to the cytoplasm as is further discussed in more detail below. On the other hand, MHC-II presented peptides will typically arise from the endosomal and lysosomal compartment via degradation and processing by acidic proteases (e.g., legumain, cathepsin L and cathepsin S) prior to delivery to the cell membrane. Thus, expression of the epitopes intended for MHC-II presentation will generally be directed to the endosomal and lysosomal compartment as is also discussed in more detail below.

In most preferred aspects, signal peptides may be used for trafficking to the endosomal and lysosomal compartment, or for retention in the cytoplasmic space. For example, where the peptide is to be exported to the endosomal and lysosomal compartment targeting presequences and the internal targeting peptides can be employed. The presequences of the targeting peptide are preferably added to the N-terminus and comprise between 6-136 basic and hydrophobic amino acids. In case of peroxisomal targeting, the targeting sequence may be at the C-terminus. Other signals (e.g., signal patches) may be used and include sequence elements that are separate in the peptide sequence and become functional upon proper peptide folding. In addition, protein modifications like glycosylations can induce targeting. Among other suitable targeting signals, the inventors contemplate peroxisome targeting signal 1 (PTS1), a C-terminal tripeptide, and peroxisome targeting signal 2 (PTS2), which is a nonapeptide located near the N-terminus. In addition, sorting of proteins to endosomes and lysosomes may also be mediated by signals within the cytosolic domains of the proteins, typically comprising short, linear sequences. Some signals are referred to as tyrosine-based sorting signals and conform to the NPXY or YXXØ consensus motifs. Other signals known as dileucine-based signals fit [DE]XXXL[LI] or DXXLL consensus motifs. All of these signals are recognized by components of protein coats peripherally associated with the cytosolic face of membranes. YXXØ and [DE]XXXL[LI] signals are recognized with characteristic fine specificity by the adaptor protein (AP) complexes AP-1, AP-2, AP-3, and AP-4, whereas DXXLL signals are recognized by another family of adaptors known as GGAs. Also FYVE domain can be added, which has been associated with vacuolar protein sorting and endosome function. In still further aspects, endosomal compartments can also be targeted using human CD1 tail sequences (see e.g., Immunology, 122, 522-531).

Trafficking to or retention in the cytosolic compartment may not necessarily require one or more specific sequence elements. However, in at least some aspects, N- or C-terminal cytoplasmic retention signals may be added, including a membrane-anchored protein or a membrane anchor domain of a membrane-anchored protein. For example, membrane-anchored proteins include SNAP-25, syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, and NOTCH.

In yet further contemplated aspects, protein turnover can be further accelerated by suitable choice of the N-terminal amino acid of the recombinant antigen or neoepitope, and it is especially preferred that the N-terminal amino acid is a destabilizing amino acid. Thus, suitable N-terminal amino acids especially include Arg, His, Ile, Leu, Lys, Phe, Trp, and Tyr, and to some degree also Asn Asp, Gln, and Glu. Such amino acids may be added to peptides targeted to the MHC-I as well as MHC-II presentation pathways. Consequently, addressing the peptides to the appropriate compartments with suitable signal sequences, and optionally modifying the peptides with destabilizing N-terminal amino acids, will help increase antigen cascading and epitope spread.

In yet further contemplated aspects, it should be noted that the various neoepitopes may be arranged in numerous manners, and that a transcription or translation unit may have concatemeric arrangement of multiple epitopes, typically separated by short linkers (e.g., flexible linkers having between 4 and 20 amino acids), which may further include protease cleavage sites. Such concatemers may have between 1 and 20 neoepitopes (typically limited by size of recombinant nucleic acid that can be delivered via a virus), and it should be noted that the concatemers may be identical for delivery to the MHC-I and MHC-II complex, or different.

Therefore, it should be appreciated that various peptides can be routed to specific cellular compartments to so achieve preferential or even specific presentation via MHC-I and/or MHC-II. Viewed from another perspective, it should be recognized that tumor associated antigens and neoepitopes may be presented via both presentation pathways, or selectively to one or another pathway at the same time or in subsequent rounds of treatment.

Consequently, as the (neo)antigens are presented via MHC-I and/or MHC-II pathways of the dendritic cells (and other antigen presenting cells), it should be recognized that processing through the immune system after administration of the activated immune competent cells to the patient will result in continued stimulation of both CD8+ and CD4+ cells in the patient, which will lead to formation of trained B-cells for formation of IgG1 as well as trained NK cells and corresponding memory cells. In addition, it should be noted that the IgG1 molecules will also enable tumor specific action by NK cells.

While not limiting to the inventive subject matter, it is generally preferred that neoepitope sequences are configured as a tandem minigene (e.g., aa12-neoepitope12-aa12), or as single transcriptional unit, which may or may not be translated to a chimeric protein. Thus, it should be appreciated that the epitopes can be presented as monomers, multimers, individually or concatemeric, or as hybrid sequences with N- and/or C-terminal peptides as already discussed above. Most typically, it is preferred that the nucleic acid sequence is back-translated using suitable codon usage to accommodate the virus and/or host codon preference. However, alternate codon usage or non-matched codon usage is also deemed appropriate.

Additionally, it is preferred that the viral delivery vehicle (or other expression construct) also encodes at least one, more typically at least two, eve more typically at least three, and most typically at least four co-stimulatory molecules to enhance the interaction between the infected dendritic (or otherwise antigen presenting) cells and immune competent cells (e.g., T-cells, NK cells, etc.). For example, suitable co-stimulatory molecules include ICAM-1 (CD54), ICOS-L, and LFA-3 (CD58), especially in combination with B7.1 (CD80) and/or B7.2 (CD86). Further contemplated co-stimulatory molecules include 4-1BBL, CD30L, CD40, CD40L, CD48, CD70, CD112, CD155, GITRL, OX40L, and TL1A. Moreover, it should be appreciated that expression of the co-stimulatory molecules will preferably be coordinated such that the antigens and/or neoepitopes are presented along with one or more co-stimulatory molecules. Thus, it is typically contemplated that the co-stimulatory molecules are produced from a single transcript using an internal ribosome entry site or 2A sequence, or from multiple transcripts. Alternatively, co-stimulatory molecules may also be delivered via separate RNA constructs.

Additionally, but not necessarily, it is contemplated that the viral vector (or other expression construct, preferably RNA) may also include a sequence portion that encodes one or more polypeptide ligands that bind to a checkpoint receptor. Most typically, binding will inhibit or at least reduce signaling via the receptor, and particularly contemplated receptors include CTLA-4 (especially for CD8+ cells) PD-1 (especially for CD4+ cells). For example, polypeptide binders can include antibody fragments and especially scFv, but also small molecule peptide ligands that specifically bind to the receptors. Once more, it should be appreciated that expression of the (poly)peptide molecules will preferably be coordinated such that the antigens and/or neoepitopes are presented along with one or more (poly)peptide molecules. Thus, it is typically contemplated that the (poly)peptide molecules are produced from a single transcript using an internal ribosome entry site or 2A sequence, or from multiple transcripts. Alternatively, and as already noted above, the immune checkpoint inhibitors may be administered to the patient before or during administration of the activated immune competent cells.

In further contemplated aspects, the expression vector or RNA may also encode include functionally associated proteins that are known to interact and provide enhancement to an immune response. For example, the expression vector or RNA may include segments that encode CD27 and CD70, CD40 and CD40L, OX40L and OX40, GITRL and GITR, IL-2 and CD122, CD137 and TRAF2, and/or ICOSL and ICOS. Likewise, suitable expression vectors and RNA may also encode include ligands that interact with inhibitory systems to provide a further enhancement to an immune response. For example, suitable (naturally occurring or engineered) ligands include a ligand that inhibits CD276/B7-H3 inhibition of T-cell activation, a ligand that inhibits B7-H4/VTCN1 inhibition of T-cell activation, a ligand that inhibits CD272/HVEM inhibition of T-cell activation, a ligand (e.g., MHC-II, etc.) that inhibits LAGS inhibition of T-cell activation, a ligand (e.g., PD-L1) that inhibits PD-1 inhibition of T-cell activation, a ligand (e.g., biologic, soluble CD28, etc.) that inhibits CTLA-4 inhibition of T-cell activation, a ligand (e.g., galectin-9, biologic, antibody, etc.) that inhibits TIM-3 inhibition of T-cell activation, a ligand (e.g., antibody, etc.) that inhibits VISTA inhibition of T-cell activation, and/or a ligand (e.g., antibody, biologic etc.) that inhibits MIC inhibition of NK cells.

Most typically, expression of the recombinant genes is driven from constitutively active regulatory sequences. However, in other aspects of the inventive subject matter, the regulatory sequences may be inducible, preferably in a selective manner using one or more regulatory signals endogenous to the cancerous tissue or synthetic inducers. In most cases, it is further preferred that the transcript will includes an IRES (internal ribosome entry site) or a 2A sequence (cleavable 2A-like peptide sequence) to again allow for coordinated expression of the cytokines and co-stimulatory molecules.

With respect to transfection of the dendritic or other antigen presenting cells, it should be noted that the recombinant nucleic acids may be administered as naked or complexed DNA (e.g., using lipofection), but it is generally preferred that the recombinant nucleic acid is part of a viral genome or a recombinant RNA. The so genetically modified virus can then be used to infect the dendritic cells in vitro, which will significantly reduce potential issues with immunogenicity of the viral vehicle. With respect to recombinant viruses it is contemplated that all known manners of making recombinant viruses are deemed suitable for use herein, however, especially preferred viruses are those already established in therapy, including adenoviruses, adeno-associated viruses, alphaviruses, herpes viruses, lentiviruses, etc. Among other appropriate choices, adenoviruses are particularly preferred. Moreover, it is further generally preferred that the virus is a replication deficient and non- immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Most typically, the desired nucleic acid sequences (for expression from virus infected cells) are under the control of appropriate regulatory elements well known in the art.

In view of the above, it should therefore be appreciated that compositions and methods presented are not only suitable for directing virally expressed antigens specifically to one or another (or both) MHC systems, but will also provide increased stimulatory effect on the CD8+ and/or CD4+ cells via inclusion of various co-stimulatory molecules (e.g., ICAM-1 (CD54), ICOS-L, LFA-3 (CD58), and at least one of B7.1 (CD80) and B7.2 (CD86)), and via secretion or membrane bound presentation of checkpoint inhibitors.

Moreover, and with respect to contemplated neoepitopes, it should be appreciated that the neoepitopes need not necessarily be expressed by the antigen presenting cells, but that at least some (or all) of the neoepitopes may also be delivered into the antigen presenting cells as individual peptides or as a polypeptide. As will be readily appreciated, such polypeptides may be synthetic peptides, or peptides that were produced in a recombinant expression system such as a bacterial and/or yeast expression system. Therefore, suitable peptides may be ‘minimal’ peptides (i.e., have a length that does not exceed the number of residues needed for binding and presentation by MHC-I or MHC-II), or have additional sequence portions at the N- and/or C-terminus. For example, additional amino acids may be present to facilitate or trigger processing or routing in the proteasome or TAP system, or to increase affinity to the MHC-I or MHC-II. Alternatively, or additionally, the additional sequence portions may also be spacer elements having preferably low to no immunogenicity and rigid secondary structures. For example, contemplated spacer portions may be useful between at least two covalently coupled neoepitopes. On the other hand, additional sequence portions may also have a functional role, and especially contemplated functional roles include detectability (e.g., via GFP portion), ability to purify (e.g., via avidin portion), or signaling function.

Where the dendritic cells or other antigen presenting cells are genetically modified to express or are exposed to antigen peptides, it should be noted that the genetic modification or exposure to the antigen peptides can be performed before contacting the dendritic cells or other antigen presenting cells with the immune competent cells. Alternatively, genetic modification or exposure may also be performed while the dendritic cells or other antigen presenting cells are in contact with the immune competent cells.

In further contemplated aspects, it is preferred that the exposed or transfected antigen presenting cells (e.g., from the patient) are incubated in vitro with the patient's immune competent cells for a time sufficient to allow instruction or activation of the immune competent cells by the antigen presenting cells, typically at least 2 hours, more typically at least 4 hours, and most typically at least 8 hours. As used herein, the terms “co-culturing” and “incubating” are synonymously used and denote a process in which the cells are maintained in a viable state that may also include cell division. Most typically, suitable ratios of antigen presenting cells (e.g., previously transfected with nucleic acid encoding a tumor related antigen or exposed to tumor related antigen) to immune competent cells are typically between 10⁴:1 (antigen presenting cells to immune competent cells) and 1:10⁴ (antigen presenting cells to immune competent cells), or between 10³:1 and 1:10³, or between 10²:1 and 1:10², or 10:1 and 1:10. However, in less preferred aspects, the exposed or transfected antigen presenting cells (e.g., from the patient) may also be incubated with the patient's immune competent cells in vivo.

In still further contemplated aspects, it is noted that instead of using the isolated dendritic cells (other isolated antigen presenting cells), the patient's bulk white blood cells (WBCs) could be cultured with the neoepitopes or transfected with nucleic acids encoding neoepitopes for expression. Such an approach is expected to cause production of desired MHC/neoepitope complexes by the antigen presenting cells in the bulk WBCs. Thus, the patient's macrophages, dendritic cells, and B-Cells provide instruction to the NK cells and T-cells so that they take on the desired properties to target the diseased tissue.

Moreover, it should be appreciated that the mixture of transfected antigen presenting cells and immune competent cells may be performed in the presence of one or more immune stimulatory cytokines. For example, suitable cytokines include IL-2, IL-7, IL-12, IL-15, and especially modified IL-15 (e.g., IL-15 superagonist from Altor Bioscience). Additionally, or alternatively, the mixture of transfected antigen presenting cells and immune competent cells may be performed in the presence of one or more ligands of pattern recognition receptors such as TLR ligands (e.g., TLR2, TLR3, TLR4, TLRS, TLR7/8, TLR9, TLR13, etc.), NLR ligands (e.g., NOD1, NOD2, etc.), RLR ligands (e.g., 5′ppp-dsRNA, Poly(dA:dT, etc.), CLR ligands (e.g., HKCA, lichenan, beta glucan peptide, etc.), and/or STING ligands (e.g., cyclic dinucleotides such as 2′2′-cGAMP, 2′3′-cGAMP, c-di-AMP, etc.).

It is further contemplated that the mixture of transfected antigen presenting cells and immune competent cells may be processed to remove one or more components before administration to the patient. For example, the mixture may be processed to remove one or more of the immune stimulatory cytokines, pattern recognition ligands, and/or dendritic or otherwise antigen presenting cells. Consequently, it should be noted that a cell-containing transfusion composition will typically include the transfected antigen presenting cells and/or the immune competent cells from the patient, possibly in further combination with expression vector or an viral delivery vehicle (e.g., adenovirus) containing a recombinant nucleic acid containing a sequence encoding one or more neoepitopes, and/or one or more neoepitope peptides. In addition, the transfusion composition may also include immune stimulatory cytokines and/or checkpoint inhibitors. Furthermore, processing of the mixture of transfected antigen presenting cells and immune competent cells may also include a step of removing exhausted T cells or a step of activating exhausted T cells. For example, the mixture may be contacted with effective quantities of antibodies against PD-L1, TIM3, LAGS, CTLA4, or CD244, or with IL21.

Where desired, the transfusion composition may also include heterologous NK cells, and particularly NK cells that are genetically modified to exhibit less inhibition. Of course, contemplated NK cells may also be administered to the patient before or after administration of the transfusion composition.

For example, the genetically modified NK cell may be a NK-92 derivative that is modified to have a reduced or abolished expression of at least one killer cell immunoglobulin-like receptor (KIR), which will render such cells constitutively activated. Of course, it should be noted that one or more KIRs may be deleted or that their expression may be suppressed (e.g., via miRNA, siRNA, etc.), including KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest as aNK cells (‘activated natural killer cells).

In another preferred aspect of the inventive subject matter, the genetically engineered NK cell may also be an NK-92 derivative that is modified to express the high-affinity Fcγ receptor (CD16). Sequences for high-affinity variants of the Fcγ receptor are well known in the art, and all manners of generating and expression are deemed suitable for use herein. Expression of such receptor is believed to allow specific targeting of tumor cells using antibodies produced by the patient in response to the treatment contemplated herein, or that are specific to a patient's tumor cells (e.g., neoepitopes), a particular tumor type (e.g., her2neu, PSA, PSMA, etc.), or that are associated with cancer (e.g., CEA-CAM). Advantageously, such cells may be commercially obtained from NantKwest as haNK cells (‘high-affinity natural killer cells).

Alternatively, the genetically engineered NK cell may also be genetically engineered to express a chimeric T-cell receptor. In especially preferred aspects, the chimeric T-cell receptor will have an scFv portion or other ectodomain with binding specificity against a tumor associated antigen, a tumor specific antigen, and/or a cancer neoepitope. As before, such cells may be commercially obtained from NantKwest as taNK cells (‘target-activated natural killer cells’) and further modified as desired. Where the cells have a chimeric T-cell receptor engineered to have affinity towards a cancer associated antigen or neoepitope, it is contemplated that all known cancer associated antigens and neoepitopes are considered appropriate for use. For example, tumor associated antigens include CEA, MUC-1, CYPB1, PSA, Her-2, PSA, brachyury, etc.

In addition, and as noted above, it is contemplated that prophylactic or therapeutic administration of the cell containing transfusion composition may be accompanied by co-administration with one or more immune checkpoint inhibitors, especially where the recombinant virus or RNA does not include nucleic acid sequences encoding polypeptides that target the checkpoint receptors. For example, especially preferred check point inhibitors include currently available inhibitors (e.g., pembrolizumab, nivolumab, ipilimumab).

Of course, it should be recognized that contemplated compositions and methods may not only be used in a single therapeutic event, but that the compositions may be administered to the patient repeatedly over time. Such repeated administration is particularly advantageous where the patient is surveyed for newly arisen neoepitopes as could be expected. These newly identified neoepitopes can then be brought to bear on modifying contemplated therapeutic compositions to better suit the patient's disease or adapt the tumor's attempt to evade attack by the immune system.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Finally, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C. . . and N, the text should be interpreted as requiring only one element from the group, not A plus N, or B plus N, etc.

What is claimed is:

1. A method of treating a patient having a tumor, comprising:
administering to the patient a plurality of immune competent cells that were previously ex vivo exposed to transfected antigen-presenting cells;
wherein the antigen-presenting cells were transfected with at least one patient-specific tumor neoepitope or an RNA or expression vector comprising a nucleic acid sequence that encodes the at least one patient-specific tumor neoepitope; and
wherein the immune competent cells are obtained from the patient having the tumor.

2. (canceled)

3. The method of claim 1 wherein the plurality of immune competent cells are enriched in at least one of a CD4+ T-cell, a CD8+ T-cell, an NK cell, a macrophage, a monocyte, and a B- cell.

4-7. (canceled)

8. The method of claim 1 wherein the at least one patient-specific tumor neoepitope is an HLA-matched patient-specific tumor neoepitope.

9. The method of claim 1 wherein the at least patient-specific tumor neoepitope further comprises a targeting sequence that targets the patient-specific tumor neoepitope to MHC-I or MHC-II presentation.

10. (canceled)

11. The method of claim 1 wherein the antigen-presenting cells were further transfected with or exposed to at least one of an immune stimulating molecule, a nucleic acid encoding at least one immune stimulating molecule, a checkpiont inhibitor, or a nucleic acid encoding at least one checkpoint inhibitor.

12-18. (canceled)

19. The method of claim 1 wherein the plurality of immune competent cells were exposed to the transfected antigen-presenting cells in the presence of a cytokine.

20. (canceled)

21. The method of claim 1 further comprising a step of administering to the patient an immune checkpoint inhibitor before the step of administering the plurality of immune competent cells.

22-23. (canceled)

24. A method of ex vivo activating immune competent cells from a patient having a tumor, comprising:
obtaining from the patient a plurality of immune competent cells;
transfecting ex vivo a plurality of antigen-presenting cells with at least one patient-specific tumor neoepitope or with an expression vector comprising a nucleic acid that encodes the at least one patient-specific tumor neoepitope; and
co-culturing the plurality of immune competent cells with the plurality of transfected antigen-presenting cells for a time sufficient to activate the immune competent cells.

25. (canceled)

26. The method of claim 24 wherein the plurality of immune competent cells are enriched in at least one of a CD4+ T-cell, a CD8+ T-cell, an NK cell, a macrophage, a monocyte, and a B-cell.

27-30. (canceled)

31. The method of claim wherein the at least one patient-specific tumor neoepitope is an HLA-matched patient-specific tumor neoepitope.

32. The method of claim 24 wherein the at least one patient-specific tumor neoepitope further comprises a targeting sequence that targets the tumor-related epitope to MHC-I or MHC-II presentation.

33. (canceled)

34. The method of claim 24 wherein the antigen-presenting cells were further transfected with or exposed to at least one of an immune stimulating molecule, a nucleic acid encoding at least one immune stimulating molecule, a checkpoint inhibitor, or a nucleic acid encoding at least one checkpoint inhibitor.

35-41. (canceled)

42. The method of claim 24 wherein the step of co-culturing is performed in the presence of a cytokine.

43-44. (canceled)

45. A pharmaceutical composition, comprising:
a pharmaceutically acceptable carrier for transfusion in combination with a plurality of immune competent cells and a plurality of transfected antigen-presenting cells;
wherein the antigen-presenting cells are cells transfected with at least one patient-specific tumor neoepitope or an expression vector comprising a nucleic acid that encodes the at least one patient-specific tumor neoepitope; and
wherein the immune competent cells are obtained from the patient having the tumor.

46. (canceled)

47. The composition of claim 45 wherein the plurality of immune competent cells are enriched in at least one of a CD4+ T-cell, a CD8+ T-cell, an NK cell, a macrophage, a monocyte, and a B-cell.

48-51. (canceled)

52. The composition of claim 45 wherein the at least one patient-specific tumor neoepitope is an HLA-matched tumor-related epitope.

53. The composition of claim 45 wherein the at least one patient-specific tumor neoepitope further comprises a targeting sequence that targets the tumor-related epitope to MHC-I or MHC-II presentation.

54. (canceled)

55. The composition of claim 45 wherein the antigen-presenting cells were further transfected with or exposed to at least one of an immune stimulating molecule, a nucleic acid encoding at least one immune stimulating molecule, a checkpoint inhibitor, or a nucleic acid encoding at least one checkpoint inhibitor.

56-62. (canceled)

63. The composition of claim 45 further comprising a cytokine or an immune checkpoint inhibitor.

64. The composition of claim 63 wherein cytokine is IL-2, IL-7, IL-12, IL-15, or a IL-15 superagonist.

65-85. (canceled)
